(19) United States
(12) Patent Application Publication
(10) Pub. No.: US 2006/0173715 A1
(43) Pub. Date: Aug. 3, 2006

(54) Health information system and method

(76) Inventor: Hao Wang, Worcester, MA (US)

Correspondence Address: EPSTEIN BECKER & GREEN, P.C., 1227 25TH STREET, N.W. 7TH FLOOR, WASHINGTON, DC, 20037, US

(21) Appl. No.: 11/341,459

(22) Filed: Jan. 30, 2006

Related U.S. Application Data
(60) Provisional application No. 60/648,429, filed on Feb. 1, 2005.

(51) Int. Cl. G06Q 10/00; A61B 5/00

(52) U.S. Cl. 705/2; 600/300

(57) ABSTRACT

The present invention relates to a distributed computer-based decision support technology for the healthcare industry. It also relates to a systematic knowledge diffusion technology for the healthcare industry. The tool and methodology packages and distributes computation and data processing software components in both centralized and federated fashions to process medical data in-situ and in real-time, applying the knowledge and best practices that can reflect the most recent advancement of medical sciences. This tool and methodology interacts with a healthcare organization's existing internal and external information sources, including those sources of its trading partners, such as practice management systems, health information systems, electronic medical records systems, lab systems, medical reference systems, and existing decision support systems. Due to this sharing of information and the ability to construct longitudinal medical records that was previously not possible, the quality of decision support can be rapidly improved, benchmarked, and standardized across the industry.

### RELATED APPLICATION

This patent application claims priority to U.S. Provisional Application Ser. No. 60/648,429 filed Feb. 1, 2005, which is incorporated herein by reference in its entirety.

### FIELD OF THE INVENTION

The present invention relates to health information technology. Specifically, the present invention relates to computer based decision support and knowledge diffusion in the health care arena.

### BACKGROUND OF THE INVENTION

Medical decisions are often either based upon incomplete patient health information and/or delayed awaiting the receipt of such information. Patient health information can include for example, a patient's medical and/or familial history. Existing decision support tools and technologies in the healthcare industry are generally confined within individual organization's boundaries and/or limited to a subset of patient data that the individual organization owns or has access to. These factors result in medical decisions made upon insufficient information; medical decisions made by local experts with knowledge confined by the individual organization's limited human capital. It is often the case that the medical decisions made for the same patient by different personnel or organizations are different, reflecting a diminished standard of medical care due to the slowness of the knowledge diffusion.

At the system level, existing decision support tools and technologies are usually purchased and customized by individual healthcare organizations, therefore, even though certain industry standards can be used as guidelines, there are variation across independent implementations. The quality of each decision support mechanism is not to be uniform therefore the effect on patient safety and healthcare quality can vary from one decision support system to another.

Additionally, current decision support systems usually do not reflect the advancement of computer science. For example, natural language processing (NLP) is not widely available in healthcare decision support tools. Such natural language processing refers to a subfield of artificial intelligence and linguistics, which processes and manipulates human languages in either spoken or written form to make computers “understand” statements in human languages

Given the complexity of medical science and technology, as well as the regulations around medical practice and medical health information, diffusion of new knowledge can be very slow. The industry lacks tools and technologies to facilitate medical knowledge dissemination in a systematic and automatic fashion. Thus, it can take quite some time to for advances in medical science and technology to become standard practice.

The healthcare industry is in need of adaptive and scalable new technologies to facilitate and automate knowledge transfer and thereby enhance the quality of medical care. The healthcare industry is also in need of adaptive and scalable new technologies to help decision makers to obtain maximum amount of information about patients' health. What is needed is real-time decision support based on aggregated patient health data from diverse sources and across organizational boundaries. Preferably this would include a patient's complete longitudinal health records. A longitudinal health or medical record is a health record comprising patient's health data accumulated over the patient's lifetime, spanning space and time, resulting in a complete historical accounts of a patient's health information. What is also needed is such decision support to be made by applying systematically and automatically the newest advancement of medical science and best practices to these longitudinal health records.

### SUMMARY OF THE INVENTION

It is an object of the invention to provide a mechanism for processing real-time medical data for patients thereby yielding medical decision support in real-time. The medical decision support refers to using computerized information systems to support decision making in the medical fields.

It is a further object of the invention to process patient's longitudinal data, acquired from diverse data sources, across organizational boundaries, and to thereby provide medical decision support.

It is a further object of the invention to provide a means for the healthcare industry to incorporate new medical knowledge into medical best practice, in a systematic and automatic fashion.

It is a further object of the invention to capture possible adverse drug events (ADEs), near miss medical malpractices, threshold events for chronological diseases, and other medical events that warrant immediate attention from the patients themselves, their doctors, and/or their associated healthcare organizations.

It is a further object of the invention to generate alert messages and securely route these messages to appropriate parties who can take appropriate actions to mitigate the risk of ADEs and near misses, to prevent further degradation of the patient's health conditions, to coordinate for better care, and to manage chronicle conditions for the patients.

It is a further object of the invention to provide a federated data collection and dissemination system, wherein such system is a distributed system having minimal central authority. Preferably the system comprises a number of smaller heterogeneous systems, with each smaller system maintaining its autonomy. Preferably the system is regional and encompasses all members of the healthcare industry, thereby providing unbiased and cross organizational studies and comparisons.

It is a further object of the invention to provide a real time data collection system that can monitor and report upon patient safety, care quality, and/or operation efficiency.

It is a further object of the invention to provide a benchmarking mechanism that can provide all organizations a reference system to align themselves with the industry's leading performers and industry standards.

The above and other features and advantages are achieved through the use of a novel health information system and method as herein disclosed. There has thus been outlined, rather broadly, the more important features of the invention in order that the detailed description thereof that follows may be better understood, and in order that the present contribution to the art may be better appreciated. There are, of course, additional features of the invention that will be described further hereinafter.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced or carried out in various ways. Also, it is to be understood that the phraseology and terminology employed herein are for the purpose of description and should not be regarded as limiting.

As such, those skilled in the art will appreciate that the conception upon which this disclosure is based may readily be utilized as a basis for the designing of other structures, methods and systems for carrying out the several purposes of the present invention. It is important, therefore, that equivalent constructions insofar as they do not depart from the spirit and scope of the present invention, are included in the present invention.

For a better understanding of the invention, its operating advantages and the specific objects attained by its uses, reference should be had to the accompanying drawings and descriptive matter which illustrate preferred embodiments of the invention.

### BRIEF DESCRIPTION OF THE DRAWINGS

FIG. 1 illustrates the concept of distributed decision support and knowledge diffusion.

FIG. 2 illustrates a high level component diagram for the system and methods.

FIG. 3 illustrates centralized operation of the system and methods.

FIG. 4 illustrates federated operation of the system and methods.

### DETAILED DESCRIPTION OF THE PREFERRED EMBODIMENTS

The invention disclosed herein is a distributed computer-based decision support and knowledge diffusion technology for healthcare industry. The tool and methodology packages and distributes computation and data processing software components in both centralized and federated fashions to process the medical data in-situ and in real-time, behind an healthcare organization's security boundary, interacting with healthcare organization's existing internal and external data sources, including the information systems belong to both the organization itself and its trading partners, such as the practice management systems (PMS), health information systems (HIS), electronic medical records systems (EMRs), lab systems, medical reference systems, and existing decision support systems. Such PMS, HIS, EMR, lab systems, medical reference systems and existing decision support systems are well known to those of ordinary skill in the art. Such data processing includes any computational processes that goes through predefined sequences of operations on the data and converts such data into useful information.

As used herein, a decision-support system or decision-support technology, also referred to as a “data mining system” or a “knowledge discovery in data system”, is any system, typically a computer-based system, which can be trained on data to classify the input data and then subsequently used with new input data to make decisions based on the training data. These systems include, but are not limited, expert systems, fuzzy logic, non-linear regression analysis, multivariate analysis, decision tree classifiers, Bayesian belief networks and, as exemplified herein, neural networks. Data mining, also known as knowledge-discovery in databases, is the practice of automatically searching large stores of data for patterns. To do this, data mining uses computational techniques from statistics, machine learning and pattern recognition

Members of the healthcare industry who might utilize this invention include, but are not limited to, hospitals, clinics, HMOs, self-insured employers groups, third-party administrators, physician groups, physician networks, pharmacy groups, pharmacy networks, or other health care program analysts or insurance carriers. Individual users may include physicians, nurses, pharmacists, customer service representatives, clerks, telephone operators, or any other person.

The host environment of the present invention will contain a mechanism to configure and interact with an entity's external trading partners. This mechanism will actively request and passively receive data from both internal and external data sources. Once new medical data is received, the tool will facilitate analysis of the data, extraction of conclusions, and reconciliation of the data with a patient's medical records in situ, correlation of the data with patient's medical history and possible problems, and acquisition of additional data from both internal and external sources if necessary. A medical record is a systematic documentation of a patient's medical history and care. It refers to the body of information which comprises the patient's health history. As used herein, reconciliation means the new data is properly categorized, grouped, and filed with old data. The tool may contain business rules and clinical logics that may be applied to the data by the rules engine to determine whether additional data is required. More specifically, the tool may apply business or clinical information such as clinical guidelines, rules, algorithms, operating protocols, and/or procedures to help the user identify recommended forms of treatment, medications, or courses of action. Pharmaceutical information such as prescription drug side effects and complications that may be associated with particular drugs or a combination of drugs and health benefit information such as insurance company rules, member information, and benefit plan resources may also be included in the tool. The clinical guidelines may cover a multitude of medical symptoms, conditions, procedures and topics, and they may include general information about effective and appropriate prescription and over-the-counter medications. The NCPDP Telecommunications Standard Format manual, including the standard format for the electronic submission of third party drug and/or medical claims, is hereby incorporated by reference. This report may be obtained, for example, from the National Council for Prescription Drug Programs, Inc., Phoenix, Arizona.

After a longitudinal medical record is reconstructed, the tool will fire off the predefined healthcare business logics pertinent to the patient(s) to process such data, and derive certain decision support results. Such results can be as minor as necessary reminders for patients and doctors to conduct certain routine care activities. Such results can be as major as possible adverse drug events (ADEs), adverse events, near miss medical malpractices, threshold events for chronological diseases, and other medical events that warrant immediate attention from the patients themselves, their doctors, and their associated healthcare organizations. Adverse drug events are adverse events involving medication use. ADEs include expected adverse drug reactions or side effects, as well as events due to error. For example, a serious allergic reaction to penicillin in a patient with no prior such history is an ADE, but so is the same reaction in a patient who does have a known allergy history but receives penicillin due to a prescribing oversight. (Definition of ADE by AHRQ). Adverse event is any injury caused by medical care. It refers to an undesirable clinical outcome resulted from some aspect of diagnosis or therapy. Near miss medical malpractices refer to events or situations that did not result in patient injury, but only because fortuitous and timely intervention, for example, a nurse happens to realize that a physician wrote an order in the wrong chart. Threshold events for chronicle diseases usually refer to certain medical measures such as lab values cross over certain predefined levels or threshold values so that certain medical diagnosis become apparent.

The tool comprises medical logic container(s) that will allow definition, configuration, and maintenance of a variety of decision support techniques such as pattern recognition, correlation, natural language processing, etc. Such pattern recognition refers to a field within the area of machine learning, which is the act of classifying data based on either prior knowledge or statistical information. Such decision support techniques and subcomponents provide real-time, in-situ analysis based on the patient longitudinal health data. Such data may include, for example, medical, including diagnostic tests or assays such as imaging and radiology tests including immunoassays, chemical assays, nucleic acid assays, calorimetric assays, fluorometric assays, chemiluminescent and bioluminescent assays, electrocardiograms, X-rays and other such tests, pharmaceutical, demographic, psychographic, and health benefit information. The systems and methods process patient data, particularly data from point of care diagnostic tests or assays, and provide an indication of a medical condition or risk or absence thereof. Rules within the medical logic container(s) can be programmed and or updated based on recent advances in medical science. These rules can be disseminated across the global computer network.

Where indicated, the tool will generate and distribute alert messages to stakeholders. A message is an object of communication, the information sent from a source to a receiver. In a preferred embodiment, such alert messages will be sent through secure channel through a computer network and follow-up and necessary escalation will be closely monitored. The tool may also automatically generate selected reports or other types of messages such as written patient information, drug information, prescription refill reminders, prescription renewal reminders, potential risks, follow-up instructions, referral information, patient allergies, risk assessments, etc.

It should be understood that the messages/reports may be delivered via one or more, preferably secure, output devices, which may be connected to the computer-based system. Examples of output devices include, but are not limited to, a cathode ray lube (CRT) display, liquid crystal displays (LCD), printers, and communication devices such as a modem, cable and audio output. It should also be understood that one or more input devices may be connected to the computer system. The data may be input into the tool via one or more input devices.

Examples of input devices include a keyboard, keypad, track ball, mouse, pen and tablet, communication device, audio input and scanner. It should be understood the invention is not limited to the particular input or output devices used in combination with the computer system or to those described herein.

The present invention provides, at the system level for the healthcare industry, a mechanism to process real-time medical data for patients to capture possible adverse drug events (ADEs), adverse events, near miss medical malpractices, threshold events for chronological diseases, and other medical events that warrant immediate attention from the patients themselves, their doctors, and their associated healthcare organizations.

The technology of the present invention enables distributed computer-based decision support for healthcare industry. It packages and distributes computation and data processing software and technologies in both centralized and federated fashions to process the medical data in-situ and in real-time, without requiring end users purchase and install these software and technologies. In one embodiment, such computation and data processing software and technologies are incorporated within the present invention. In a second embodiment, the present invention incorporates such software and technologies that exist within a user's system. The present invention provides for the aggregation of patient's longitudinal health data from a variety of data sources, including data sources from multiple organizations, to be processed along with patient's new medical data. Historical data is thus taken into consideration along with new medical events to offer better decision support for physicians as well as the patients.

The technology of the present invention comprises secure messaging services to route the information such as alerts to appropriate parties who can take appropriate actions to mitigate the risk of ADEs and near misses, and to prevent further degradation of the patients' health conditions.

The present invention also enables distributed disease management and case management for modem health care industry. New medical data for patients are processed in real-time to capture any events that need medical attention.

In essence, the present invention is a federated decision support system that provides tight integration with all relevant internal and external data sources so that a patient's longitudinal data can be collected in real time to appropriate decisions regarding patient care can be derived. As used in this application, “real time” refers to the operation time of such data collection tasks being in the realm of a few minutes. Being a federated decision support system, the present invention shares approved medical logics widely and in a uniform fashion. Such medical logics can reflect the newest advancement of medical science and best practices. Healthcare organizations can reap benefits of sharing standard business logics across the distributed systems and not to be limited to individual implementation and development of these logics. Due to this sharing of information, the quality of decision support can be benchmarked and standardized across the industry. Also, because it provides a federated decision support system, the present invention will increase the speed of dissemination and application of new advancements of medical science and technology. Any node in the federated network can be delegated to translate the new knowledge thereby providing an immediate transition from basic science to practice. The methods of practice are then diffused across the networks so all organizations in the network can take advantage of the new medical knowledge and apply it to their practices immediately.

The present invention further comprises a medical event aware automated processing system. This system automatically captures medical events that warrant immediate intervention.

The present invention further comprises a messaging capable federated network, to ensure rapid healthcare intervention by generating, routing, and managing secure messages that can be sent to all stakeholders in the system, including government for public health concerns and other reasons. There are several existing messaging technologies known to those of ordinary skill in the art, which could serve as a messaging engine. Examples of such technology include but are not limited to IBM MQ Series, Microsoft MQ, Java JMS; ant the like.

There are several existing business integration technologies known by those of ordinary skill in the art, which could serve as the medical data receptacle and environment for the tool's process orchestration. Examples of such technology include but are not limited to Microsoft BizTalk Server 2004, IBM Business Integration Server; Seebeyond eGate; Novell eXtend; ant the like.

There are several existing web service hosting and development technologies known by those of ordinary skill in the art, which could serve as the web service hosting environment. Examples of such technology include but are not limited to Microsoft .NET technology including IIS web server and Visual Studio .NET development environment; IBM WebSphere software platform and WebSphere Studio development environment; and the like.

There are several existing database technologies known by those of ordinary skill in the art. Examples of such technology include but are not limited to Microsoft SQL Server 2000, IBM DB2, Oracle 9i, and the like.

There are several existing computation technologies for pattern recognition, natural language processing, data mining, etc., available in the market and known by one of ordinary skill in the art. Examples of pattern recognition technology include but are not limited to Matlab and neural networks; examples of natural language processing include but are not limited to Answer Anywhere and Alice; examples of data mining include but are not limited to SPSS, SAS, and other OLAP tools such as Microsoft SQL Server OLAP, Oracle OLAP, LBM DB2 OLAP.

The present invention further comprises an event driven object execution environment, object wrapping, transaction management, and workflow, collectively called the data processing engine. The present invention further comprises a user interface to create, manage, configure, and execute medical logics; a user interface to manage, configure, synchronize, update; databases to correlate medical logics with individual patients and a user interface to manage these correlations; databases to audit, log, and archive transactions; and alert generation and presentation mechanism.

It should be understood that the invention is not limited to a particular technology, computer platform, particular processor, particular high-level programming language or Web service. Additionally, the computer system of the present invention may be a multiprocessor computer system or may include multiple computers connected over a computer network.

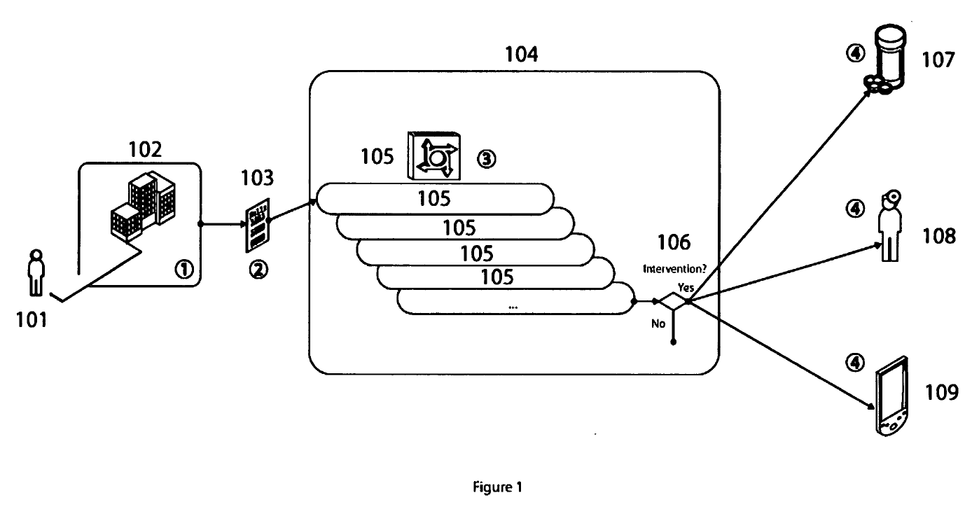

FIG. 1 illustrates the high level concept of the present invention in a local health information infrastructure. As illustrated, the tool monitors the events relating to patient 101 that visits provider organization 102 resulting in clinical message/medical data 103 containing the medical information generated from the visit. Such data monitoring tool provides continuous supervision of a patient's health data without continuous attendance. Clinical message/medical data 103 is collected into data processing system 104 hosting the computational software components for clinical logics for the present invention. Data processing system 104 comprises modules of medical logic object(s) 105 including but not limited to natural language processing, pattern recognition, decision support logistics, and data mining. In this embodiment the tool and the data processing system 104 is installed in a local health information infrastructure (LHII), and actively monitors clinical message/medical data 103 for patient 101. Once the tool receives data, a series of intelligent medical logic object(s) 105 are executed to the medical records. If interventions are deemed necessary by the clinical rules 106, instant secure messages are generated and routed to all relevant members such as the patient's pharmacy benefit management parties (PBMs) 107, the patient's primary care physicians (PCPs) provider 108, and patient 109.

In practice, the following steps occur: 1) patient 101 visits provider organization 102 and receives treatment and/or tests; 2) as a result of this treatment and/or tests the patient's medical record is modified, and the modification sent to the tool in way of clinical message/medical data 103; 3) information pertaining to this new medical record is provided to the tools of the present invention such as data processing system 104, medical logic object(s) 105, and clinical rules 106, the information is then processed for retrieval at a later date and/or examined in view of related information to identify potential adverse drug events and/or other conditions requiring immediate notice; and 4) where indicated, alert signals are generated and submitted as appropriate.

As the clinical logics executed to the medical information are separated from particular practitioner or provider organization 102, the expertise reflected from this clinical logics is not limited to the current knowledge base in provider organization 102. Therefore, the new advancement of the medical knowledge and best practices can be programmed and realized in these medical logic object(s) 105. Because the alert messages are subsequently generated and routed back to provider 108 and other members, the consultation by the independent source provided by the present invention can be given to provider 108 so that the new knowledge can be automatically revealed. Knowledge diffusion thus can happen in an automatic and systematic fashion.

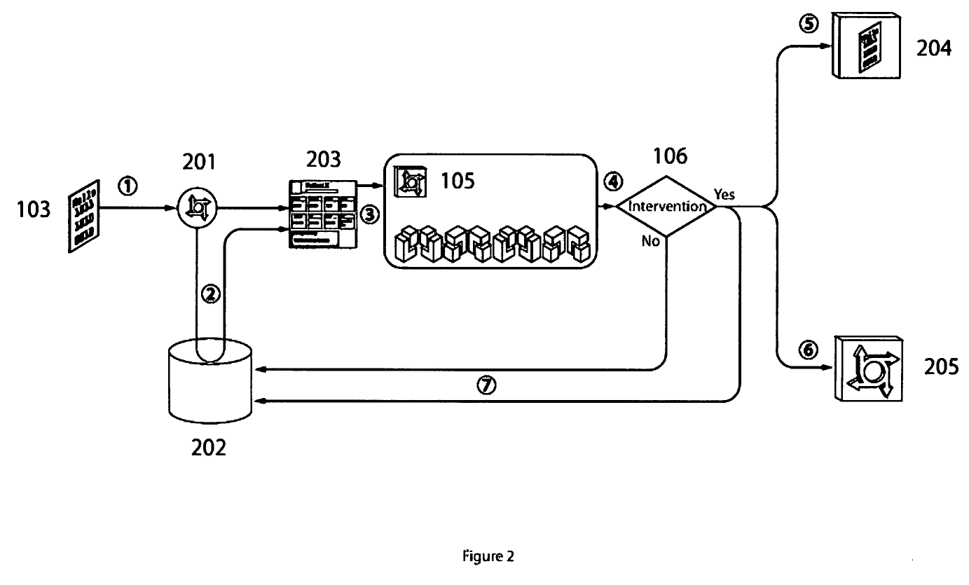

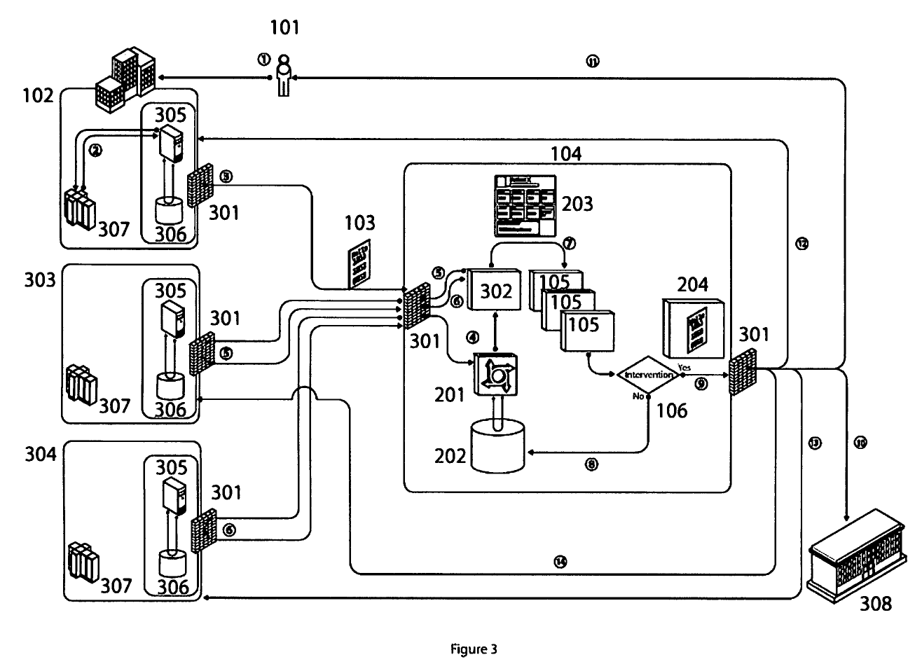

FIG. 2 illustrates the logical components of the tool of the present invention. As shown, the tool comprises receiving Web services 201, federated database 202, longitudinal medical record 203, medical logic object(s) 105 such as pattern recognition tool, clinical rules 106, message engine(s) 204 for generating and routing alerts, and set of Web services 205 for presenting the result, routing the information from a knowledgebase, and retaining the logging information and reports. Federated database 202 is a type of a metadata database which transparently integrates multiple autonomous database systems into a single uniform virtual database for use to store and retrieve data. In practice, data pertaining to clinical message/medical data 103 is passed to Web services 201, which serves as a receiving module. By way of the federated database embodied in the U.S. patent application Ser. No. 11/117,499, the tool queries related data set forth in the patient's longitudinal medical record 203. Data obtained from longitudinal medical record 203 is then provided to the tool. Medical and health care logic steps are applied to the complete record by medical logic object(s) 105 such as clinical pattern recognition tool and clinical rules 106. Should the medical and health care logic steps indicate intervention, message engine(s) 204 and Web services 205 are initiated. Data resulting from the medical and health care logic steps and the complete record is then saved in the federated database 202, which is illustrated in details in the U.S. patent application Ser. No. 11/117,499, as well as in FIG. 3.

FIG. 3 is a sample implementation of the tool of the present invention in a centralized fashion, where a local health information infrastructure decides to host the medical logic centrally and thus becomes data processing system 104. As shown, clinical message/medical data 103 for a patient 101 who visits the provider organization 102 enrolled in the data processing system 104 is copied and routed to data processing system 104 for processing, across the organization's security firewall 301. The centralized engine, also behind its own security firewall 301, contains a pre-processing module or data aggregator 302, which is used to obtain patient data from multiple organizations such as first organization 303 and second organization 304 in addition to provider organization 102. Data aggregator 302 then forms longitudinal medical record 203 for the patient and passes this longitudinal medical record 203 to medical logic object(s) 105 and clinical rules 106, determining whether to intervene. In practice, patient 101 is treated by provider organization 102 which in turn generates clinical message/medical data 103 for patient 101 and sends the new data to data processing system 104. Data processing system 104 then retrieves complete medical data for the patient from all providers, payers, and ancillary facilities such as first organization 303 and second organization 304 to construct longitudinal medical record 203. This longitudinal medical record 203 is then fed to the engine where medical logic object(s) 105 and clinical rules 106 pertinent to the patient are run against this complete medical record, complete with the new information from the hospital. After processing this medical record, the engine then decides whether intervention is deemed necessary. If it is necessary, the engine will dispatch secure messages via message engine(s) 204 to relevant parties such as provider organization 102, patient 101, and pharmacy benefit management 308.

FIG. 3 also briefly illustrates how a longitudinal medical record is constructed from a federated database infrastructure as described in U.S. patent application Ser. No. 11/117,499. Multiple organizations form a regional alliance to share medical data and install standard gateway server 305 and staging database 306 which interact securely with the organizations'backend systems 307. Then data aggregator 302 receives a new medical data resulted from a medical event for patient 101 for provider organization 102; data aggregator 302 dispatches data queries to other organizations containing medical information for patient 101. Then the data aggregator collects the data returned from these organizations and forms longitudinal medical record 203 for patient 101.

The ability to generate a complete longitudinal medical record 203 for patient 101 from multiple organizations such as provider organization 102, first organization 303 and second organization 304 further advances the medical best practice and knowledge diffusion of medical science. Without this ability, many medical best practices can not be easily put in practical use due to lack of information. The present invention builds on the federated data sharing infrastructure as described in the U.S. patent application Ser. No. 11/117,499, utilizing the tools containing new knowledge, medical logic object(s) 105 and clinical rules 106, can offer more comprehensive automatic consultation for patient 101 and provide this consultation to the parties responsible for the patient's health.

It is not required that the tools and processing engines are installed centrally. In a different embodiment, the processing engines and tools in this invention can be installed in any organizations so that the processing logics and speed can be fine tuned to suit the needs of an independent organization. This embodiment provides for a quicker response that is more pertinent to the local organization. Additionally, several organizations can act as each other's backup tool to achieve resilience and redundancy.

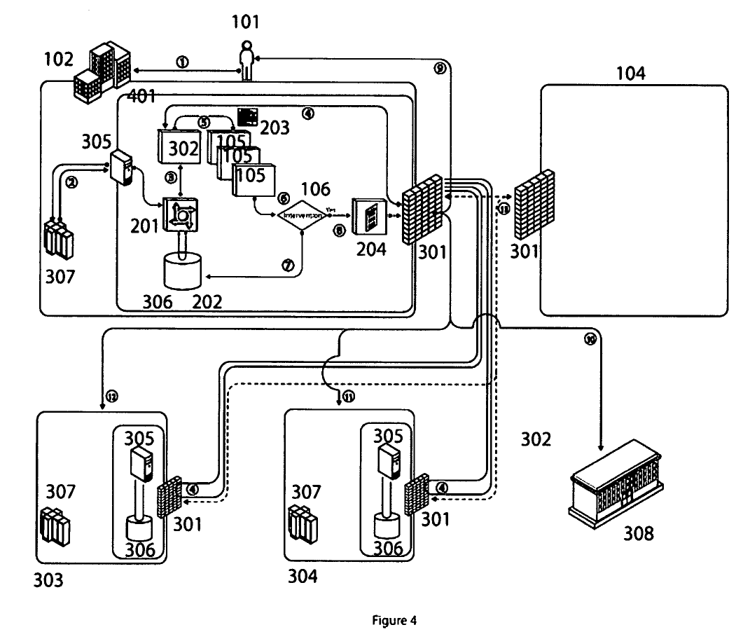

FIG. 4 is a sample implementation tool of the present invention in a federated fashion in a hospital setting. In this embodiment, medical logic object(s) 105 and clinical rules 106 are installed in one provider organization 102. Data aggregator 302, secure message engine(s) 204 are also installed in provider organization 102. When patient 101 visits provider organization 102, the medical event triggers off data aggregator 302. Data aggregator 302 dispatches data query to first organization 303 and second organization 304 of the provider organization 102, collects medical data for patient 101 from first organization 303 and second organization 304, and forms the longitudinal medical record 203. Then the medical logic object(s) 105 and clinical rules 106 are applied to this longitudinal medical record 203. If a decision to intervene is made, secure message engine(s) 204 is used to route alerts and consultation results to parties responsible for the patient's health. Please note all these operations, except the distributed queries and final alert messaging, are happening behind security firewall 301 of provider organization 102.

In practice, patient 101 is treated by provider organization 102 such as a hospital which in turn generates new medical data for the patient and sends the new data to local engine 401. Local engine 401 then uses data aggregator 302 to retrieve the complete medical data for the patient from all providers, payers, and ancillary facilities such as first organization 303 and second organization 304 to construct longitudinal medical record 203. This longitudinal medical record 203 is then passed to medical logic object(s) 105 and clinical rules 106 where clinical logics pertinent to the patient are run against this longitudinal medical record 203 with the new information from the hospital. After processing this longitudinal medical record 203, the local engine determines whether intervention is deemed necessary. If it is necessary, local engine 401 will use message engine(s) 204 to dispatch secure messages to relevant parties such as the primary care physician, patient 101, and pharmacy benefit management 308.

Eventually, by continued use of the present system, the result codes database will grow in the amount of information available and the appropriateness of various studies with respect to actual patient outcome will become more apparent. Feedback provided will advise physicians that certain studies are not likely to provide them with valuable information and physician behavior will gradually change by eliminating unnecessary tests. Use of this system can enable enforcement as well as behavior modification to occur. As a result, this managed utilization of various diagnostic studies can reduce costs.

Having now described a few embodiments of the invention, it should be apparent to those skilled in the art that the foregoing is merely illustrative and not limiting, having been presented by way of example only. Numerous modifications and other embodiments are within the scope of one of ordinary skill in the art and are contemplated as falling within the scope of the invention and any equivalent thereto. It can be appreciated that variations to the present invention would be readily apparent to those skilled in the art, and the present invention is intended to include those alternatives. Further, since numerous modifications will readily occur 20 to those skilled in the art, it is not desired to limit the invention to the exact construction and operation illustrated and described, and accordingly, all suitable modifications and equivalents may be resorted to, falling within the scope of the invention.

What is claimed is:

1. An information system comprising:
a monitoring tool, wherein said monitoring tool monitors data related to a patient visit, and wherein said monitoring tool generates a message, said message comprising information related to said patient visit;
a data processing system, wherein said processing system receives said message, wherein said processing system comprises clinical logics, wherein said processing system applies said clinical logics to said message and determines whether intervention is necessary or whether intervention is not necessary, and when intervention is necessary, said processing system generates and routes alert messages to third parties.

2. The information system of claim 1, wherein said clinical logics comprise at least one of natural language processing, pattern recognition, medical decision support, or data mining

3. The information system of claim 1, wherein third parties comprise at least one of patient pharmacy benefit management parties, patient primary care physicians, or patient.

4. The information system of claim 1, wherein said clinical logics comprise at least one of Web-based information retrieval tool, federated database, longitudinal medical record, pattern recognition tool, messaging engine, information presentation tool, knowledge base management tool, and reporting tool.

5. The information system of claim 1, wherein said monitoring tool monitors information in real-time.

6. The information system of claim 2, wherein said clinical logics further comprise at least one of a database comprising possible adverse drug events, a database comprising near miss medical malpractices, or a database comprising threshold events for chronological diseases.

7. The information system of claim 1, wherein said intervention messages are sent via a secure channel through a computer network.

8. The information system of claim 1, wherein such system is a distributed system.

9. The information system of claim 1, further comprising a data collection module, wherein said data collection module collects data relates to patient safety, care quality or operation efficiency.

10. The information system of claim 9, further comprising a data reporting module, wherein said data reporting module provides reports comprising data related to patient safety, care quality or operation efficiency.

11. The information system of claim 10, further comprising a benchmarking mechanism.

12. A method of information tracking comprising:
copying medical data specific to a patient and routing said medical data to a data processing system,
processing said medical data within said data processing system,
augmenting said medical data with additional patient specific data, said additional patient specific data obtained by way of a pre-processing module or data aggregator, said augmented medical data and said additional patient specific data to comprise patient specific longitudinal medical record;
passing said patient specific longitudinal record to at least one processing module and to at least one medical logic module; and
determining whether intervention is required.

13. The method of information tracking of claim 12, further comprising:
if intervention is required, preparing an alert message; and
routing said alert message, wherein said routing is mediated via a messaging engine.

14. The method of information tracking of claim 12, wherein said medical logic module comprises at least one of a database comprising possible adverse drug events, a database comprising near miss medical malpractices, or a database comprising threshold events for chronological diseases.

15. The method of information tracking of claim 12, wherein said alert message is routed via a secure channel through a computer network.

16. The method of information tracking of claim 12, further comprising:
collecting data, wherein said collecting data is mediated via a data collection module, and wherein said data collection module collects data related to patient safety, care quality or operation efficiency.

17. The method of information tracking of claim 16, further comprising:
reporting data, wherein said reporting data is mediated via a data reporting module, and wherein said data reporting module provides reports comprising data related to patient safety, care quality or operation efficiency.

18. The method of information tracking of claim 17, further comprising:
benchmarking data.
